(19) United States
(12) Patent Application Publication
(10) Pub. No.: US 2026/0285899 A1
(43) Pub. Date: Sep. 24, 2026

(54) METHOD OF FORMING MULTI-SPECIFIC ANTIBODIES FROM HOMODIMER ANTIBODIES AND PHARMACEUTICAL PRODUCT INCLUDING MULTI-SPECIFIC ANTIBODIES THUS OBTAINED

(71) Applicant: Pfizer Inc., New York, NY (US)

(72) Inventors: Verzhiniya AHO, Andover, MA (US); Arch David CREASY, Andover, MA (US); Aaron Michael D’ANTONA, Cambridge, MA (US); Arnab DE, Andover, MA (US); Jacob Roy DONAHUE, Cambridge, MA (US); Timothy ISKRA, Andover, MA (US); Ryan Andrew JACKOBEK, Andover, MA (US); Michael KING, Andover, MA (US)

(21) Appl. No.: 19/472,746

(22) PCT Filed: Apr. 4, 2024
(86) PCT No.: PCT/IB2024/053294

§ 371 Date: Oct. 6, 2025

Related U.S. Application Data
(60) Provisional application No. 63/494,817, filed on Apr. 7, 2023.

(51) Int. Cl. C07K 1/107; C07K 1/04; C07K 1/10

(52) U.S. Cl. CPC C07K 1/1075; C07K 1/042; C07K 1/10

(57) ABSTRACT

The invention relates to a method of forming multi-specific antibodies from first and second homodimer antibodies comprising the steps of: —providing at least one solution including the homodimer antibodies; —loading said at least one solution onto an adsorbent chromatography medium, whereby the homodimer antibodies bind to the medium; —contacting the bound antibodies with a reductant; and —eluting the bound antibodies with an elution buffer thereby obtaining an eluate pool. The method further comprises, after the step of contacting the bound antibodies with a reductant, a step of contacting the bound or eluted antibodies with an oxidant, whereby multi-specific antibodies are formed from first and second homodimer antibodies. The invention is also directed to a pharmaceutical product including multi-specific antibodies obtained by such a method.

### TECHNICAL FIELD

The present invention relates to processes of manufacturing multi-specific antibodies and more specifically to in-vitro methods of forming multi-specific antibodies from first and second homodimer antibodies.

### BACKGROUND OF THE INVENTION

Multi-specific antibodies are important and novel class of therapeutics that have the potential to demonstrate improved potency and efficacy compared to current monoclonal/monospecific based therapies.

Several in vitro methods for generating multispecific antibodies have been explored, that utilize amino acid point mutations to promote the formation of heterodimerized antibodies from two unique homodimers. For example, U.S. Pat. No. 9,527,926 discloses a method wherein point mutations of opposite charges are introduced into the hinge regions and/or the CH3 domain of two homodimeric antibodies. With this method, a high ratio of heterodimerization during a redox reaction to produce a heterodimerized antibody can be achieved.

Such method can be implemented in a purification process wherein each homodimer is produced in a respective bioreactor for cell culture and subjected to initial purification steps, including an affinity capture step, and then jointly pooled in a tank for heterodimerization by a redox reaction. A method of this type is illustrated on FIG. 1.

Such methods are relatively complex in that they involve two separate bioreactor feed streams, a dedicated tank for the redox reaction, and an intermediate ultrafiltration/diafiltration operation to remove excess reductant.

There is therefore a need for an improved method for generating multispecific antibodies that eliminates the need for separate bioreactors, intermediate steps and equipment such as tanks. It is also desirable to provide a method that achieves a high degree of heterodimerization, that do not damage, or significantly reduce, the biological activity of the protein.

### SUMMARY OF THE INVENTION

The inventors have found in particular that the redox reaction may advantageously be achieved during the affinity capture step.

A method is described below that addresses the aforementioned problem.

According to a first aspect of the present invention, the method of forming multi-specific antibodies from first and second homodimer antibodies comprising the steps of:
providing at least one solution including the first and second homodimer antibodies;
loading said at least one solution onto an adsorbent chromatography medium, whereby the first and second homodimer antibodies bind to the medium;
contacting the bound antibodies with a reductant; and
eluting the bound antibodies with an elution buffer thereby obtaining an eluate pool,
   wherein the method further comprises, after the step of contacting the bound antibodies with a reductant, a step of contacting the bound or eluted antibodies with an oxidant, whereby multi-specific antibodies are formed from first and second homodimer antibodies.

With the method thus defined, the formation of multispecific antibodies is simplified, the manufacturing costs are decreased and the speed of manufacturing is increased. The product quality and yield are also improved due to the higher heterodimerization efficiency that can be achieved.

The method of the invention may further include one or more of the following features:
the medium is a protein A chromatography medium;
the elution buffer comprises the oxidant, whereby the bound antibodies are contacted by the oxidant during the elution step;
the method further comprises, before the elution step, a step of washing the medium with an initial wash buffer for impurities to flow through with the wash buffer while the homodimer antibodies remain bound to the medium; and
the method further comprises, before the elution step, a step of washing the medium with a reductant wash buffer containing the reductant, whereby the bound antibodies are contacted by the reductant during said washing step.

According to a preferred embodiment, the method may further include one or more of the following features:
the reductant wash buffer further contains at least one stabilizing excipient to stabilize the homodimer antibodies binding to the medium;
the concentration of reductant in the reductant wash buffer is between 30 and 500 mM;
the pH of the reductant wash buffer is between 5 and 9; and
the method further includes, before the elution step, an additional step of washing the medium with an additional wash buffer to remove excess reductant.

According to an alternative embodiment, the elution buffer contains the reductant and the antibodies are contacted with the oxidant after the elution step.

The method of the invention may further include one or more of the following features:
the reductant is cysteine;
the contact time of the reductant with the medium is greater than 3 minutes, preferably between 48 and 72 minutes, preferably of about 1 hour;
the oxidant comprises cystine or oxidized glutathione (GSSG) or Dehydroascorbic Acid (DHA);
the oxidant comprises cystine and the concentration of cystine in the elution buffer is between 0.1 and 1 mM, preferably of about 0.8 mM;
the oxidant comprises GSSG and the concentration of GSSG in the elution buffer is between 1 and 100 mM, preferably of about 50 mM;
the contact time of the oxidant with the antibodies is greater than 1 hour;
the concentration of the homodimer antibodies loaded onto the medium is between 5 and 60 mg, preferably of about 40 mg, of antibodies per ml of medium;
the method further comprises the step of adding oxidant in the eluate pool;
the method further comprises the step of adding a neutralization buffer in the eluate pool to neutralize the eluate pool at a pH between 7 and 8.8, preferably at a pH of 8;
the percentage of multi-specific antibodies in the neutralized eluate pool is equal to or greater than 90% of the total population of antibodies;
the reductant wash buffer further contains at least one stabilizing excipient to stabilize the homodimer antibodies binding to the medium and the at least one stabilizing excipient comprises arginine or β cyclodextrin (bCDX) or a combination thereof;
the first and second homodimer antibodies contain Fc domains of a human IgG;
the multi-specific antibodies are bi-specific antibodies or tri-specific antibodies.

In a further aspect of the invention, it is provided a pharmaceutical product including multi-specific antibodies obtained by a method according to the first aspect of the invention.

### DETAILED DESCRIPTION OF THE INVENTION

The following definitions will be used in the present description and claims:
an “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g. monospecific, bispecific, trispecific, multispecific), and includes portions of intact antibodies that retain the ability to bind to a given antigen (e.g. an “antigen-binding fragment”), and any other modified configuration of an immunoglobulin molecule that comprises an antigen binding site. Examples of antibody antigen-binding fragments and modified configurations include (i) a Fab fragment (a monovalent fragment consisting of the VL, VH, CL and CH1 domains); (ii) a F(ab′) 2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883;
a “Fc domain” refers to the portion of an immunoglobulin (lg) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains. Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 20 63 (1): 78-85 and as described in Kabat et al., 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein);
a “heterodimer, “heterodimeric complex”, or “heterodimeric polypeptide” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where polypeptides in addition to the first and second polypeptide are present;
a “bispecific antibody” refers to a molecule that has binding specificity for at least two different epitopes. In some embodiments, bispecific antibodies can bind simultaneously two different antigens. In other embodiments, the two different epitopes may reside on the same antigen;
as used herein, a “trispecific antibody” is an antibody that has binding specificity for three different epitopes. In some embodiments, trispecific antibodies can bind simultaneously three different antigens. In other embodiments, the three different epitopes may reside on the same antigen;
as used herein, a “multispecific antibody” is an antibody that has binding specificity for at least two different epitopes. In some embodiments, multispecific antibodies can bind simultaneously at least two different antigens. In other embodiments, the at least two different epitopes may reside on the same antigen;
the term “harvested cell culture fluid” (or “harvested CCF” or “HCCF”) refers to a solution containing at least one target substance which is sought to be purified from other substances also present. The harvested CCFs are often complex mixtures containing many biological molecules (such as proteins, antibodies, hormones, and viruses), small molecules (such as salts, sugars, lipids, etc.) and even particulate matter. While a typical harvested CCF of biological origin may be an aqueous solution or suspension, it may also contain organic solvents used in earlier separation steps such as solvent precipitations, extractions, and the like. Examples of harvested CCFs that may contain valuable biological substances amenable to the purification by various embodiments of the present invention include, but are not limited to, a culture supernatant from a bioreactor, a homogenized cell suspension, plasma, plasma fractions, and milk;
the term “load” refers to any material containing the target substance or an intermediate constituent thereof, either derived from the cell culture (the harvested CCF) or from a chromatography step (thus partially purified), and loaded onto a chromatography medium;
the term “load challenge” refers to the total mass of substance loaded onto the chromatography medium in the load cycle of a chromatography step, measured in units of mass of substance per unit volume of medium;
the term “impurities” refers to materials in the harvested CCF that are different from the protein of interest (or target protein) and are desirably excluded from the final therapeutic protein formulation. Typical impurities include nucleic acids, proteins (including HCPs, high and low molecular weight species, peptides, endotoxins, viruses) and small molecules;
the term “excipients” means the constituents of the final therapeutic protein formulation, which are not the therapeutic protein. The excipients typically include protein stabilizers, surfactants, amino-acids e.g. contributing to protein stabilization, etc. . . . ;
unless stated otherwise, the term “about” associated with a numeral value means within a range of +10% of said value.

### BRIEF DESCRIPTION OF THE FIGURES

The invention will now be further illustrated by the following Example, with reference to the Figures listed below, wherein:

FIG. 1 is a schematic illustration of an example of prior art process steps for the formation and the purification of a multi-specific antibody;

FIG. 2 is a schematic illustration of an example of process steps for the formation and the purification of a multi-specific antibody according to the invention;

FIG. 3 illustrates the effect of reductant concentration, in a first example of bispecific antibody (Example 1) and associated operating conditions, in a method according to the invention;

FIG. 4 illustrates the effect of reductant concentration, in a second example of bispecific antibody (Example 2) and associated operating conditions, in a method according to the invention; and

FIG. 5 illustrates the effect of reductant concentration, in an example of trispecific antibody (Example 3) and associated operating conditions, in a method according to the invention.

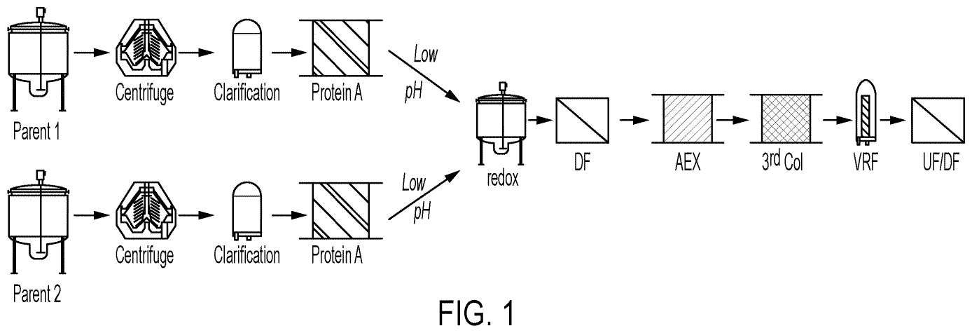

FIG. 1 illustrates a conventional process for the formation and purification of multi-specific antibodies, in particular bi-specific antibodies, from parent homodimers of two different types. In the illustrated method, each parent homodimer is produced in a respective bioreactor for cell culture. The first steps of purification are also achieved by centrifugation of the HCCF from the respective bioreactor, clarification and capture chromatography (more specifically, in the illustrated example, protein A chromatography) steps. Those steps are performed in parallel with respective equipment dedicated to each parent homodimer.

Each clarified CCF is loaded into the respective protein A chromatography column (or more generally onto a protein A chromatography medium), in conditions such that both parent homodimer antibodies (or homodimers) bind to the protein A medium. The medium is then washed with one or more wash buffers such that impurities bound to the medium are removed. The parent homodimers are then eluted from the medium at low pH conditions by means of an elution buffer, whereby the eluate from each protein A column contains the respective parent homodimers, which are then loaded into a redox tank.

This tank is dedicated to the redox reaction, whereby the heterodimers are formed from the parent homodimers.

The solution containing the heterodimers is then subjected to further purification steps, namely in the illustrated example: a diafiltration step (DF), an anion exchange chromatography step (AEX), an additional chromatography step (e.g. a cation exchange chromatography step), a virus filtration step and a final ultra-filtration/dia-filtration step (UFDF).

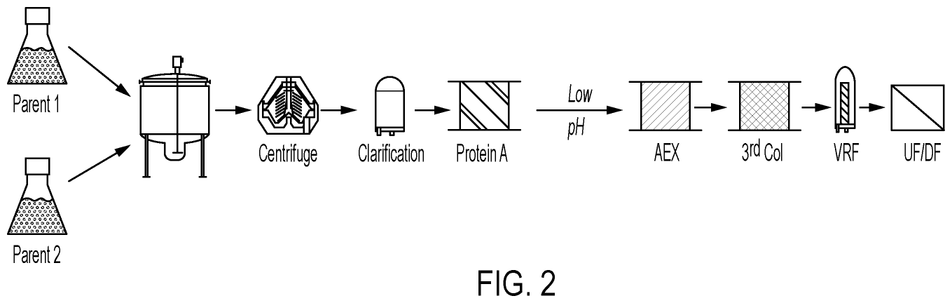

As represented on FIG. 2, in a method according to the invention both parent homodimers (first and second homodimers) may be produced in a single bioreactor, or alternatively in two separate bioreactors (not shown), and jointly pooled in a single HCCF subjected to initial steps of purification. As for the prior art example, those initial steps include centrifugation and clarification that can be achieved in a single line, with pieces of equipment which are common to both parent homodimers.

The solution defined by the CCF thus clarified and containing both parent homodimers (the first and second homodimers) is then loaded into a single protein A chromatography column, or more generally onto a protein A chromatography medium, or still more generally onto an adsorbent chromatography medium, in conditions such that both parent homodimers bind to the medium.

The medium is then washed with one or more wash buffers (initial wash buffers) such that impurities bound to the medium flow through with the wash buffer(s) and are therefore removed, while the parent homodimers remain bound to the medium. The medium is then washed with a further wash buffer containing a reducing agent (or reductant) in the preferred embodiment. The excess reductant is then removed from the column by washing the medium with an additional wash buffer.

The bound antibodies are then eluted from the medium at low pH conditions by means of an elution buffer that contains an oxidant, whereby multi-specific antibodies are formed from the parent homodimers in the elution pool. The eluate pool collected from the protein A column contains the thus formed multi-specific antibodies (heterodimers).

The conditions in which the heterodimers are formed on this adsorbent chromatography medium, in particular on the protein A chromatography column, will be discussed in the foregoing.

Like in the conventional process of FIG. 1, the solution (or eluate pool) containing the heterodimers is then subjected to further purification steps, namely in the illustrated example: a diafiltration step (DF), an anion exchange chromatography step (AEX), an additional chromatography step (e.g. a cation exchange chromatography step), a virus filtration step and a final ultra-filtration/dia-filtration step (UFDF).

### Example 1

The invention will now be further illustrated by this Example 1, corresponding to a method of forming a bispecific antibody A, according to the invention, in the context of a process illustrated on FIG. 2. The Example is provided for illustrative purpose only and should not be construed as limiting the scope of the invention.

In this illustrative Example, the bispecific antibody is made from two parent human IgG2 homodimers A1, A2.

In this Example, the parent homodimers are loaded into a protein A chromatography column, which is in that case the adsorbent chromatography medium, and processed as outlined in Table 1 below.

The protein A resin used in this Example is more specifically MabSelect SuRe LX® since it is commonly used in the downstream processing of monoclonal antibodies due to its high product capacity, caustic stability and capability to remove process related impurities (e.g. host cell proteins and DNA). Other protein A resins commercially available could alternatively be used. More generally, other adsorbents, such as membranes, may be used. That may apply to other adsorbents including other interaction modalities, e.g. ion exchange, hydrophobic interaction, and IMAC, that bind antibodies.

The column is first equilibrated with 5 column volumes (CV) of equilibration buffer (50 mM Tris, 150 mM NaCl, pH 7.5) before the column is loaded with the clarified CCF containing both of the parent homodimers. After loading is complete, the column is washed again with the equilibration buffer (wash 1) to remove unbound material, followed by a second wash (wash 2) of 50 mM Tris, 0.5 M CaCl₂), PH 7.5 for 5 CV to remove any weakly bound impurities. A third wash (wash 3) of 10 mM Tris, 10 mM NaCl, pH 7.5 is applied for 3 CV to remove any calcium chloride before applying a fourth wash buffer including a reductant (wash 4) for 5 CV. In that case, the reductant is cysteine and the fourth wash buffer includes 50 mM Tris, 34 mM cysteine at pH 8.1. The reductant wash reduces the disulfide bonds in the hinge region between the two heavy chains of the bound antibodies. Next, the reductant is removed from the column with another wash (wash 5) of 10 mM Tris, 10 mM NaCl, pH 7.5 leaving the reduced antibodies bound to the column. Finally, a low pH elution buffer containing an oxidant is applied for 5 CV to elute the antibodies from the affinity column. In that case, the oxidant is cystine and the elution buffer comprises 150 mM Glycine, 0.8 mM cystine, at pH 3.50.

The eluted antibodies are collected into a pool where they begin to heterodimerize into the multi-specific and reform the disulfide bonds in the hinge region of the product. The column is then striped, sanitized, and stored.

| | | | Linear |
|---|---|---|---|
| Step | | | Velocity |
| Description | Solution Description | CVs¹ | (cm/hr)² |
| Equilibration | 50 mM Tris, 150 mM NaCl, pH 7.5 | 5 | ≤300 |
| Load | HCCF | N/A | ≤240 |
| Wash 1 | 50 mM Tris, 150 mM NaCl, pH 7.5 | 2 | ≤240 |
| Wash 2 | 50 mM Tris, 0.5M CaCl₂, pH 7.5 | 5 | ≤300 |
| Wash 3 | 10 mM Tris, 10 mM NaCl, pH 7.5 | 3 | ≤300 |
| Wash 4 | 50 mM Tris, 34 mM Cysteine, pH 8.1 | 5 | ≤120 |
| Wash 5 | 10 mM Tris, 10 mM NaCl, pH 7.5 | 5 | ≤300 |
| Elution⁵ | 150 mM Glycine, 0.8 mM Cystine pH | N/A | ≤300 |
| | 3.50 | | |
| Strip | 50 mM Glycine, 250 mM NaCl, pH 2.7 | 5 | ≤300 |
| Sanitization | 0.1N NaOH | 5 | ≤300 |
| Storage | 2% Benzyl Alcohol, 50 mM Tris, 150 | 4 | ≤300 |
| | mM NaCl, pH 7.5 | | |

In this example, the concentration of the homodimer parent antibodies loaded onto the medium (protein A resin) is 40 mg of antibodies per ml of medium (load challenge).

Also, the contact time of the wash buffer including the reductant with the medium is of about 1 hour.

Experiments have been conducted on that basis with the parent homodimers A1, A2, to evaluate the effect on the heterodimerization of variations of reductant (cysteine) concentration in the reductant wash buffer (wash 4). The other operating conditions remained the same as outlined in Table 1.

In these experiments, the concentration of cysteine in the reductant wash was varied between 0 and 55 mM and the percentage of bispecific antibodies in the eluate pool, as compared to the total amount of antibodies (homodimers and heterodimers), was measured by conventional methods such as HPLC based methods.

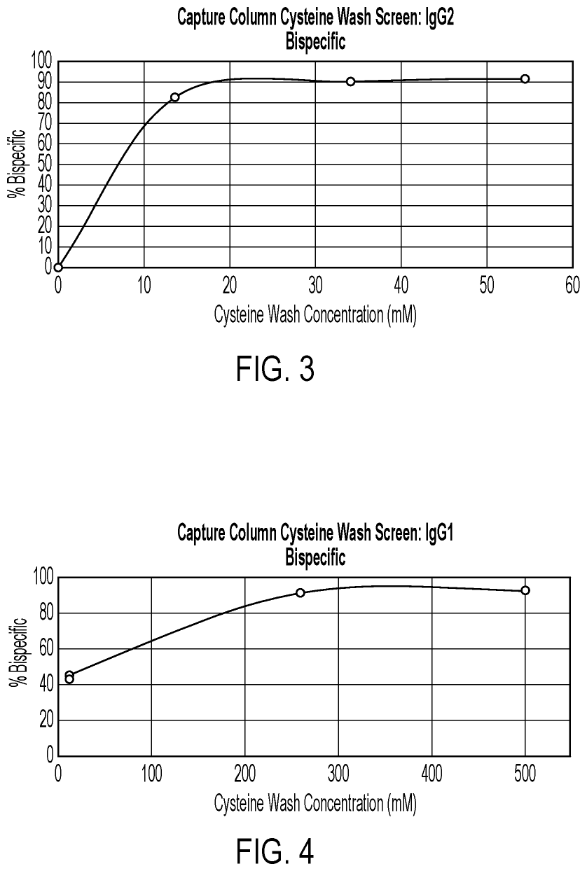

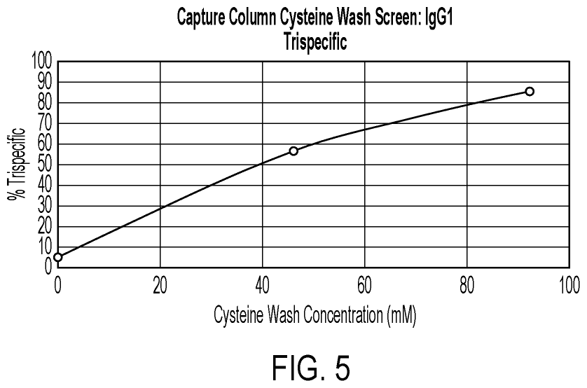

The data generated are represented on FIG. 3.

The data suggest that the efficiency of the heterodimerization (or heterodimerization ratio), as measured by the percentage of bispecific antibodies, increases almost linearly with the concentration between 0 and about 15 mM and then reaches a maximum value of efficiency of 90%. The ratio then remains roughly constant at the maximum value as the concentration increases. In particular, it may be noted that the percentage of bispecifics remains constant at 90% within the range 30-55 mM of concentration. As derived from other experiments (not reflected by FIG. 3), higher cysteine concentrations in the reductant wash result in approximately the same percentage of bispecifics.

In this Example, the experiment suggests that a cysteine concentration in the reductant wash of about 34 mM may be optimal to maximize the heterodimerization while minimizing the amount of cysteine.

While it has been established that a cysteine concentration of 34 mM may be optimal in this example, it is contemplated that the method of the invention may suitably be carried out at various cysteine concentrations, such as about 10 mM, 15 mM, 20 mM, 25 mM or between 30 and 500 mM.

### Example 2

The same experiment was run with a second pair of parent antibodies for generating a second bispecific antibody B.

In this Example 2 illustrative of the invention, the bispecific antibody is made from two parent human IgG1 homodimers B1, B2.

Similarly to Example 1, the conditions and buffer compositions outlined in Table 1 were applied, with the cysteine concentration in wash 4 (reductant wash) varied between 13.3 and 500 mM.

It can be extrapolated that the heterodimerization ratio increases almost linearly with the concentration between 13.3 and about 260 mM and then reaches a maximum value of about 95%. The ratio then remains roughly constant (between 90 and 95%) as the concentration increases. Looking at the lower concentration range, it can be expected that the heterodimerization ratio is lower than 50% when the concentration is lower than 30 mM. Such a low heterodimerization ratio is not desirable for a method of production of multi-specific antibodies, especially at a commercial scale.

In this Example, the experiment suggests that a cysteine concentration in the reductant wash of about 260 mM may be optimal to maximize the heterodimerization while minimizing the amount of cysteine, and that concentrations lower than 30 mM would not be acceptable.

### Example 3

The same experiment was run with a third pair of parent antibodies for generating a trispecific antibody C.

In this Example 3 illustrative of the invention, the trispecific antibody is made from two parent human IgG1 homodimers C1, C2.

Similarly to Examples 1 and 2, the conditions and buffer compositions outlined in Table 1 were applied, with the cysteine concentration in wash 4 (reductant wash) varied between 0 and 95 mM.

In this case, the heterodimerization ratio increases substantially linearly over the whole range of concentration to reach a maximum value of about 90% at 95 mM cysteine in the reductant wash. The heterodimerization ratio is lower than 50% when the concentration is lower than 40 mM.

In this Example, the experiment suggests that a cysteine concentration in the reductant wash of about 95 mM may be optimal to maximize the heterodimerization.

Based on Examples 1-3, it is observed that a concentration of reductant in the wash buffer within the range 30-500 mM may be suitable for forming a wide range of multi-specific antibodies with a high conversion rate.

It is however contemplated that the method of the invention may suitably be carried out at different reductant concentrations, such as about 10 mM, 15 mM, 20 mM, or 25 mM.

### Example 4

In Example 4, which is illustrative of the invention, the same parent homodimers antibodies B1, B2 as in Example 2 were subjected to the same method for forming the bispecific antibody B (as outlined in Table 1).

An experiment was conducted to evaluate the effects of contact time of the reductant wash (wash 4) on the formation of bispecific at 4 mg/ml protein load challenge. 3 minutes and 5 minutes of contact time were tested. 50 mL of homodimer combination was loaded onto a ProA capture using standard buffers with the addition of the reductant wash. The results showed a higher percentage of bispecific when a longer contact time is present: 85.6% and 87.5% for respectively 3 minutes and 5 minutes contact time.

While the experiment suggests that 3 minutes or more may be an acceptable contact time, it was found with further experiments a contact time between 48 and 72 minutes could consistently achieve a percentage greater than 90%. In particular, it was found that a good compromise between the heterodimerization ratio and the process time would be achieved by a contact time of about 1 hour.

It is however contemplated that the method of the invention may suitably be carried out with contact times such as about 5 minutes, 10 minutes, 15 minutes, 20 minutes, 25 minutes, 30 minutes, 35 minutes, 40 minutes, 45 minutes.

### Example 5

In Example 5, which is illustrative of the invention, the same parent homodimers antibodies B1, B2 as in Example 2 were subjected to the same method for forming the bispecific antibody B (as outlined in Table 1).

An experiment was conducted to evaluate the effects of load challenge on the formation of bispecific. 8 mg/mL and 40 mg/ml of homodimer proteins were loaded onto the column for this purpose at 1:1 homodimer ratio. 5 min contact time of the reductant wash was applied.

Results showed in both cases a similar bispecific conversion at 87.7%, which is also similar to the 4 mg/mL load challenge used in Example 4. This result suggests that the method of the invention can be efficiently carried out within a relatively broad range of protein amount loaded onto the column.

It was found in further experiments that such conversion rate may be achieved with a load challenge between 38 and 48 mg/mL, in particular with a load challenge of about 40 mg/mL.

### Example 6

In Example 6, which is illustrative of the invention, the same parent homodimers antibodies B1, B2 as in Example 2 were subjected to the same method for forming the bispecific antibody B (as outlined in Table 1).

An experiment was conducted to evaluate the effects of adding a wash step to remove reductant (cysteine) and its effects on bispecific formation.

In a first part of the experiment, the capture buffer sequence as outlined in Table 1 was applied, but not including the wash step (wash 5) post-reductant wash. In this case, the wash (wash 4) is directly followed by the elution of the proteins.

In a second part of the experiment, the same sequence as outlined in Table 1 was applied, thus including an additional wash (wash 5) after the reductant wash. This step allows for removing residual reductant.

The method in both scenarios achieved relatively high heterodimerization ratios (respectively 74% without additional wash and 86.8% with additional wash). However, a significant improvement of bispecific formation percentage can be seen in the experiment that had a wash following the reductant wash.

### Example 7

In Example 7, which is illustrative of the invention, the same parent homodimers antibodies B1, B2 as in Example 2 were subjected to the same method for forming the bispecific antibody B (as outlined in Table 1), save that different reductants and/or reductant concentration were tried in the reductant wash (wash4).

The experiment was conducted to evaluate the effects of alternative reductants to cysteine on the heterodimerization rate. In the experiment a 1 hour contact time for the reductant wash buffer (wash 4) was applied.

In a first part of the experiment, cysteine was used at 500 mM. The achieved heterodimerization rate was found at 87.23%.

In a second part of the experiment, TCEP (tris(2-carboxyethyl)phosphine) was used at 40 mM. The achieved heterodimerization rate was found at 89.01%.

In a third part of the experiment, glutathione was used at 40 mM. The achieved heterodimerization rate was found at 47.85%.

The experiment suggests that preferred reductants for the method of the invention are cysteine and TCEP.

While it has been established that a cysteine concentration of 500 mM or a TCEP concentration of 40 mM are suitable to achieve high heterodimerization rates, it is contemplated that the method of the invention may suitably be carried out at various concentrations of one of those reductants, such as about 10 mM, 15 mM, 20 mM, 25 mM or between 30 and 500 mM.

### Example 8

In Example 8, which is illustrative of the invention, another bispecific antibody D was formed with the method of the invention from pairs of IgG1 parent homodimers D1, D2. The same method as outlined in Table 1 was applied as baseline conditions, with some variations in the pH and composition of the reductant wash (wash 4). In particular, the effect of the presence of stabilizing modifiers at different pH on the heterodimerization rate (% conversion) was evaluated.

The results of the corresponding experiments are captured in Table 2 and discussed in the following.

TABLE 2
assembly of heterodimeric antibodies
in clarified conditioned media

| | | | | % |
|---|---|---|---|---|
| | | | | Conver- |
| | Sample Name | Wash 4 (Cys) | Wash5 | sion |
| Control | No Cys | No Cys | + | 0% |
| pH | Cys (pH 7.5) | pH 7.5 | + | 34.12% |
| | Cys (pH 8) | pH 8 | + | 78.98% |
| | Cys (pH 8.5) | pH 8.5 | + | 82.10% |
| | Cys (pH 9.0) | pH 9.0 | + | 79.73% |
| Oxidant | Cys/GSSG (pH | Cys (pH 7.5) | GSSG | 50.71% |
| | 7.5) | | | |
| Modifier/ | Cys + Arg | Cys + Arg (pH 7.5) | + | 83.22% |
| Excipient | Cys + βCDX (2HP) | βCDX (pH 7.5) | + | 85.51% |
| (pH 7.5) | Cys + βCDX + Arg | Arg + βCDX (pH | + | 86.03% |
| | (pH 7.5) | 7.5) | | |
| | Cys + αCDX (2HP) | αCDX (2HP) (pH | + | 40.74% |
| | | 7.5) | | |
| | Cys + γCDX (2HP) | γCDX (2HP) (pH | + | 57.32% |
| | | 7.5) | | |
| Excipient | Cys + Arg (pH 8) | Arg (pH 8) | + | 84.79% |
| (pH 8.0) | | | | |

The contact time for the reducing agent, as well as the stabilizers/excipients and oxidizing agent was 5 minutes. The control run was run without any L-Cysteine (Cys). The concentration of the L-Cys for al runs was 250 mM. The concentration of L-Arg used was 100 mM, and the concentration of the oxidant Glutathione Disulfide (GSSG) was 25 mM. The concentration of the A 2-hydroxy-propyl (HP) derivative of all the three α/β/γ analogs of the cyclodextrins (CDX) was used. The concentration of 2-hydroxypropyl-α-cyclodextrin or α CDX (2HP) in Table 2 was 5% (w/v). The concentration of 2-hydroxypropyl-α-cyclodextrin or a CDX (2HP) in Table 2 was 5% (w/v). The concentration of 2-hydroxypropyl-β-cyclodextrin or βCDX (2HP) in Table 2 was 20% (w/v). In the experiment using the combination of L-Arg and βCDX (2HP), the L-Arg was at a final concentration of 50 mM, while the βCDX (2HP) was at a final concentration of 10% (w/v). The concentration of 2-hydroxypropyl-γ-cyclodextrin or γCDX (2HP) in Table 2 was 20% (w/V).

In this Example 8, experiments were carried out at various pH conditions in the presence of L-Cysteine as the reducing agent (250 mM)—except for the control run—, stabilizers/excipients (amino acids, carbohydrates, etc. . . . ), and oxidizing agents (glutathione disulfide, i.e. GDS or GSSG). Wash 5 according to Table 1 is applied in all cases (+), in one experiment with an additional oxidant (GSSG).

A first experiment was carried out across a range of pH conditions (from pH 7.5 to 9.5) for the reductant wash (wash 4). It was found that pH had a drastic effect on the extent of the reaction. While the recombination at a pH of 7.5 was only 34.12%, the conversion increased to about 80% at a higher pH (8-9).

However, for proof-of-concept purpose, a pH of 7.5 was selected to study the effects of stabilizers/excipients and oxidizing agents.

In a second experiment, an oxidizing agent (GSSG) was added to the wash (wash 5) post-reductant wash (wash 4) and was found to improve the efficacy of recombination from 34.12% to nearly 50%.

In a third experiment, two different kinds of stabilizing modifiers (amino acids like L-Arginine and sugars like cyclodetrins) were studied. It was found that L-Arginine (noted ‘Arg’) could drastically increase the efficacy of recombination at both pH 7.5 (from 34.12% to ~85%) and at a pH of 8. Amongst carbohydrates, three distinct kinds of cyclodextins (α cyclodextrin: ‘aCDX’; β cyclodextrin: ‘bCDX’; and γcyclodextrin: ‘gCDX’)—which vary by their glycosidic linkages-were studied. For the cyclodextrins, the 2-hydroxy propyl derivative was used. The 2-hydroxy propyl derivatives of the cyclodextrins were found to be significantly more soluble than the cyclodextrins that did not have the derivative.

It was found that while the α cyclodextrin and γcyclodextrin could only moderately increase the efficacy from 34.12% to ~40% and ~57% respectively, β cyclodextrin could drastically increase the efficacy of the reaction at a pH of 7.5 (from 34.12% to ~85%).

A mixture of two of the most promising stabilizers (amino acid L-Arginine and a sugar β cyclodextrin) was also tried. The mixture was found to additively increase (to ~86%) the efficacy than either of the stabilizers alone. This is probably because of a combination of the different underlying modes of stability conferred by L-Arginine and β cyclodextrin.

The hypothesis at the basis of this experimental work was that the stabilizing excipients would stabilize the two homodimers and orient them optimally for the exchange (heterodimer formation). A common reducing agent (L-Cysteine) was used in order to facilitate the reduction and corresponding exchange of the homodimers. The hypothesis that stabilized protein components would lead to greater recombination efficiency at an optimal pH in the presence of reducing agent and stabilizing excipients was thus confirmed.

The pH conditions shown to be optimal for the reductant wash buffer (wash 4), in this Example and for other homodimers, are between 5 and 9 as those pH conditions were found suitable for achieving high heterodimerization rates, such as about 80% and above.

It is for example contemplated that the method of the invention may suitably be carried out at pH conditions for the reductant wash such as 5, 5.5, 6, 6.5, 7, 7.5, 8, 8.25, 8.5, 8.75, or 9.

Further experiments were conducted to evaluate the efficiency of the method according to the invention, in terms of heterodimerization ratio, when used in different conditions, including different buffer compositions, different oxidants, different oxidant concentrations, different oxidant contact time, and also different chromatography media.

### Example 9

Example 9 is illustrative of an alternative embodiment of the invention, wherein the elution buffer contains the reductant and the antibodies are contacted with the oxidant after the elution step.

In this Example, the antibody E used for the experiment is a IgG1 Bispecific (from parental homodimers E1 and E2).

Parental homodimers antibodies E1, E2 were subjected to the method as outlined in Table 3 below, at different concentrations of reductant (cysteine) in the neutralized (pH 8) elution buffer. Oxidant (GSSG) was then added after buffer exchanging out of the elution pool.

The experiment was conducted to evaluate the effects of this alternative approach to forming heterodimers on column. In the experiment, at least 1 hour contact time for the reductant (Cysteine) was applied. Load challenge was 40 mgmL.

TABLE 3

| | | | Linear |
|---|---|---|---|
| Step | | | Velocity |
| Description | Solution Description | CVs | (cm/hr) |
| Equilibration | Expi293 Expression Medium [+} | 4 | 156 |
| | GltuaMAX with known spiked protein | | |
| | (EE and RR) | | |
| Load | HCCF | N/A | 156 |
| Wash 1 | 137 mM NaCl, 8.1 mM Na2HPO4, 2.7 | 2 | 156 |
| | mM KCl, 1.47 mM KH2PO4, pH 7.2 | | |
| Elution | 150 mM Glycine, 500 mM Cysteine, | N/A | 156 |
| | pH 3.50 | | |
| Wash2 | 137 mM NaCl, 8.1 mM Na2HPO4, 2.7 | 10 | 156 |
| | mM KCl, 1.47 mM KH2PO4, pH 7.2 | | |
| Sanitization | 0.1N NaOH | 1 | 156 |
| Storage | 2% Benzyl Alcohol, 50 mM Tris, 150 | 5 | 156 |
| | mM NaCl, pH 7.5 | | |

Cysteine used at concentrations between 50 and 500 mM achieved a heterodimerization rate at 87-89%.

The experiment suggests that column redox can occur in a different approach, wherein the reductant is contained in the elution buffer and the oxidant is added after the elution step, to best fit the specific production needs. Adding an oxidant post-elution was thus found to be efficient in forming disulfide bonds.

As for the alternative chromatography media, beyond protein A as used in the above Examples, it was found that membrane chromatography could be successfully used to carry out the invention as an alternative to resin in a chromatography column. Also, HIC media with standard HIC buffers.

As for the elution buffer, it preferably includes cystine as oxidant. However, the oxidant may alternatively be oxidized glutathione (GSSG) or Dehydroascorbic Acid (DHA). The oxidant of the elution buffer may also be made of a combination of those products, whereby the elution buffer may include one or more of these products.

In case the oxidant comprises cystine, it is preferred that the concentration of cystine in the elution buffer be between 0.1 and 1 mM, preferably of about 0.8 mM, as those concentrations were found to achieve optimal conversion rates.

For the same reason, in case the oxidant comprises GSSG, it is preferred that the concentration of GSSG in the elution buffer be between 1 and 100 mM, preferably of about 50 mM.

As for the contact time of the elution buffer containing the oxidant with the antibodies, it is preferably greater than 1 hour, in order to maximize the conversion rate.

Depending on the homodimers to be converted into heterodimers and depending on the specific modalities, the method of the invention may advantageously include additional steps to enhance the conversion rate.

For example, a reductant may be added to the elution buffer for enhancing the reduction of the disulfide bonds of the homodimers. The addition of reductant in the elution may either be an additional measure to the presence of reductant in the reductant wash buffer or an alternative thereto.

Also, as an additional measure to the presence of oxidant in the elution buffer, an oxidant may be spiked in the eluate pool for enhancing the reformation of the disulfide bonds of the heterodimers.

It was also found that the neutralization of the eluate pool at a pH between 7 and 8.8, preferably at a pH of 8, was associated with an increased conversion rate. Therefore, the method of the invention may preferably further comprise a step of adding a neutralization buffer in the eluate pool to neutralize the eluate pool at the above pH values.

The method of the invention was found to be highly effective and advantageously applicable to various types of homodimers, in particular to antibodies of the sub-classes IgG1, IgG2 and IgG4. The method of the invention is suitable to achieve high conversion rates i.e. percentages of multi-specific antibodies of the total population of antibodies in the neutralized eluate pool, such as equal to or greater than 90%. The invention is highly efficient and suitable for forming multi-specific antibodies, that may be bi-specific, tri-specific antibodies or others.

1. Method of forming multi-specific antibodies from first and second homodimer antibodies comprising the steps of:
providing at least one solution including the first and second homodimer antibodies;
loading said at least one solution onto an adsorbent chromatography medium, whereby the first and second homodimer antibodies bind to the medium;
contacting the bound antibodies with a reductant; and
eluting the bound antibodies with an elution buffer thereby obtaining an eluate pool,
wherein the method further comprises, after the step of contacting the bound antibodies with a reductant, a step of contacting the bound or eluted antibodies with an oxidant, whereby multi-specific antibodies are formed from first and second homodimer antibodies.

2. Method according to claim 1, wherein the medium is a protein A chromatography medium.

3. Method according to claim 1 or 2, wherein the elution buffer comprises the oxidant, whereby the bound antibodies are contacted by the oxidant during the elution step.

4. Method according to any one of claims 1 to 3, further comprising, before the elution step, a step of washing the medium with an initial wash buffer for impurities to flow through with the wash buffer while the homodimer antibodies remain bound to the medium.

5. Method according to any one of claims 1 to 4, further comprising, before the elution step, a step of washing the medium with a reductant wash buffer containing the reductant, whereby the bound antibodies are contacted by the reductant during said washing step.

6. Method according to claim 5, wherein the reductant wash buffer further contains at least one stabilizing excipient to stabilize the homodimer antibodies binding to the medium.

7. Method according to claim 5 or 6, wherein the concentration of reductant in the reductant wash buffer is between 30 and 500 mM.

8. Method according to any one of claims 5 to 7, wherein the pH of the reductant wash buffer is between 5 and 9.

9. Method according to any one of claims 5 to 8, further including, before the elution step, an additional step of washing the medium with an additional wash buffer to remove excess reductant.

10. Method according to any one of claims 1 to 4, wherein the elution buffer contains the reductant and wherein the antibodies are contacted with the oxidant after the elution step.

11. Method according to any one of claims 1 to 10, wherein the reductant is cysteine.

12. Method according to any one of claims 1 to 11, wherein the contact time of the reductant with the medium is greater than 3 minutes, preferably between 48 and 72 minutes, preferably of about 1 hour.

13. Method according to any one of claims 1 to 12, wherein the oxidant comprises cystine or oxidized glutathione (GSSG) or Dehydroascorbic Acid (DHA).

14. Method according to claim 13, wherein the oxidant comprises cystine and the concentration of cystine in the elution buffer is between 0.1 and 1 mM, preferably of about 0.8 mM.

15. Method according to claim 13 or 14, wherein the oxidant comprises GSSG and the concentration of GSSG in the elution buffer is between 1 and 100 mM, preferably of about 50 mM.

16. Method according to any one of claims 1 to 15, wherein the contact time of the oxidant with the antibodies is greater than 1 hour.

17. Method according to any one of claims 1 to 16, wherein the concentration of the homodimer antibodies loaded onto the medium is between 5 and 60 mg, preferably of about 40 mg, of antibodies per ml of medium.

18. Method according to any one of claims 1 to 17, further comprising the step of adding oxidant in the eluate pool.

19. Method according to any one of claims 1 to 18, further comprising the step of adding a neutralization buffer in the eluate pool to neutralize the eluate pool at a pH between 7 and 8.8, preferably at a pH of 8.

20. Method according to claim 19, wherein the percentage of multi-specific antibodies in the neutralized eluate pool is equal to or greater than 90% of the total population of antibodies.

21. Method according to any one of claims 1 to 20, wherein the reductant wash buffer further contains at least one stabilizing excipient to stabilize the homodimer antibodies binding to the medium and the at least one stabilizing excipient comprises arginine or β cyclodextrin (βCDX) or a combination thereof.

22. Method according to any one of claims 1 to 21, the first and second homodimer antibodies contain Fc domains of a human IgG.

23. Method according to any one of claims 1 to 22, wherein the multi-specific antibodies are bi-specific antibodies.

24. Method according to any one of claims 1 to 22, wherein the multi-specific antibodies are tri-specific antibodies.

25. Pharmaceutical product including multi-specific antibodies obtained by a method according to any one of claims 1 to 24.
